(19) United States
(12) Patent Application Publication
(10) Pub. No.: US 2010/0055050 A1
(43) Pub. Date: Mar. 4, 2010

(54) NICOTINE CHEWING GUM ON A STICK

(76) Inventor: Kathleen Moore, Newport News, VA (US)

Correspondence Address: KATHLEEN MOORE, 20 TUKAWAY CT., NEWPORT NEWS, VA, 23601, US

(21) Appl. No.: 12/202,249

(22) Filed: Aug. 30, 2008

(51) Int. Cl. A61K 9/68; A61P 25/34

(52) U.S. Cl. 424/48

(57) ABSTRACT

A device is disclosed for helping a smoker to quit smoking. The device includes a shaft with a mass of nicotine chewing gum cooperative with one end. A smoker controls the timing and quantity of nicotine delivered by controlling the amount of gum chewed and the duration of chewing. In some embodiments partially depleted gum can be replaced on the shaft for later use. A cover can be included to protect the gum when not in use. The cover can be a simple paper wrapper or a rigid cover with a mechanism to attach it to the shaft. Portion indicators can divide the gum into separately chewable portions. The shaft can be a simple stick, or a hollow tube containing a supply of gum for delivery as needed. Some hollow tube embodiments expel gum from the tube using a sliding mechanism or a twisting mechanism that functions like a lipstick.

### FIELD OF THE INVENTION

The invention generally relates to nicotine substitutes, and more specifically to nicotine-containing chewing gum.

### BACKGROUND OF THE INVENTION

In recent years, it has become generally known that smoking and other forms of tobacco usage have a significant adverse effect on the health of tobacco users and of people exposed to “second-hand smoke” produced by smokers. Every year, thousands of people die from the diseases caused by smoking and other forms of tobacco usage (referred to generically herein as “smoking”). Regular cigarette smoking increases the concentration of nicotine and carbon monoxide in the blood stream, thereby increasing the risk of heart attack and stroke. People who smoke may develop lung cancer, throat cancer, premature wrinkles, lack of stamina, blood-shot eyes and various other lung illnesses such as smokers' cough, emphysema, and the like. Due to the increased risk of diseases and other side-effects of smoking, smokers often desire to quit smoking or to find a healthier alternative.

However, the addictive nature of nicotine generally makes it highly difficult for smokers to quit smoking. Smokers generally develop an addicted dependency that requires them to maintain a certain level of nicotine in their blood stream. One approach to providing smoking cessation treatments is therefore to provide nicotine supplements that can provide relief from nicotine withdrawal symptoms when needed. The use of such nicotine supplements is known to increase the success of smokers attempting to quit smoking.

Various products have been developed that deliver nicotine or a nicotine substitute to the blood of a smoker via the mouth, nasal cavity, or skin, so as to assist the smoker in quitting his or her smoking habit. From a behavior modification standpoint, these products are most effective if they enable a smoker to maintain control over the delivery, timing, and dosage level of the nicotine or nicotine substitute. Often, the knowledge that a quick and effective treatment for withdrawal symptoms is readily at hand reduces the anxiety of a smoker and allows the smoker to postpone and minimize the use of the product, thereby accelerating the rate of weaning and minimizing the cessation period. However, most nicotine substitution products do not provide optimal control to a smoker over the delivery, timing, and dosage level of the nicotine or nicotine substitute.

Nicotine chewing gum, for example, provides a nicotine substitute to the blood stream of a smoker through tissues within the smokers' mouth. Once chewing has commenced, however, there is often a desire not to “waste” the gum (which can be costly), and a hygienic and convenient means for storing the gum for later use is often not available. A smoker may therefore continue to chew the gum until all of the nicotine or nicotine substitute has been absorbed, whether or not the full dosage was needed. This can lead to delivery of too much nicotine or nicotine substitute, and thereby slow the weaning process and prolong the smoking cessation period.

Trans-dermal nicotine patches slowly release nicotine or nicotine substitutes into the skin. These devices do not provide oral stimulation to the user, and may cause skin irritation. Also, nicotine or nicotine substitute is delivered at a rate that is determined by the formulation and design of the patch, and is not under the control of the smoker once use of the patch has commenced. As with nicotine chewing gum, this can lead to delivery of more nicotine or nicotine substitute than is needed, thereby slowing the weaning process and prolonging the smoking cessation period.

Another approach is a nicotine spray that is sprayed into the nasal cavity. While this approach allows a smoker to control the timing and quantity of nicotine or nicotine substitute that is delivered, it is not well suited to a gradual delivery of nicotine or nicotine substitute, is generally unpleasant, and may cause nasal irritation to the smoker. Nicotine spray also fails to provide oral stimulation to the smoker. Similar disadvantages apply to compounds that are swallowed as a whole or delivered in dry powdered form into a smoker's respiratory tract.

### SUMMARY OF THE INVENTION

A device is claimed for helping a tobacco user to quit the use of tobacco. The device is effective for tobacco users who smoke tobacco, chew tobacco, use snuff, or use tobacco in any other form. The term “smoker” is used herein to refer to all users of tobacco, and the term “smoking” is used to refer to all uses of tobacco. The device includes a stick or shaft with a mass of nicotine chewing gum attached to one end of the stick. The term “nicotine chewing gum” is used herein to refer to any chewing gum that contains a smoking cessation compound, such as nicotine or a nicotine substitute, which alleviates smoking withdrawal symptoms when the gum is chewed and the smoking cessation compound is thereby absorbed into the bloodstream through tissues in the mouth.

The claimed device enables a smoker to control the timing and the amount of smoking cessation compound that is absorbed into the bloodstream. This is accomplished by enabling the smoker to “bite off” only a desired quantity of the gum, and/or by enabling the smoker to replace the gum onto the stick for later use, once the severity of nicotine cravings has subsided. In preferred embodiments, the gum is divided into segments that allow a smoker to easily bite off a desired quantity. In further preferred embodiments, a cover is provided that protects the chewing gum from contaminants while on the stick. In some of these embodiments, the cover is a simple paper wrapper, while in other embodiments the cover is a rigid cover with an attachment mechanism that attaches the cover to the stick.

In further embodiments the stick has a hollow interior filled with chewing gum, and in some of these embodiments a chewing gum delivery mechanism is included that allows chewing gum to be easily extracted from the hollow interior in desired amounts. In some of these embodiments the chewing gum delivery mechanism includes a protrusion that can be slid along the exterior of the stick, in a manner similar to the mechanism that extends the blade from the handle of a typical box-cutter. In other embodiments, the chewing gum delivery mechanism includes a twisting mechanism that functions in a manner similar to a typical lipstick.

One general aspect of the present invention is a device for helping a tobacco user, herein referred to as a “smoker,” to quit the use of tobacco, herein referred to as “smoking.” The device includes a shaft having a length and shape that allow a smoker to conveniently hold a proximal end of the shaft in one hand while placing a distal end of the shaft in smoker's mouth, and a mass of chewing gum cooperative with the distal end of the shaft. The mass of chewing gum is removable from the distal end of the shaft and contains a quantity of a smoking cessation compound in a form suitable for absorption into the smoker's bloodstream through tissues within the mouth of the smoker.

In preferred embodiments the smoking cessation compound is nicotine or a nicotine substitute. In other preferred embodiments the quantity of smoking cessation compound is at most sufficient to alleviate nicotine withdrawal symptoms to a degree comparable to a dosage of nicotine received by smoking a cigarette. In certain embodiments the smoking cessation compound is formulated and configured such that it will be absorbed over a period of time into the bloodstream of the smoker as the smoker chews the mass of chewing gum, so as to enable the smoker to limit the quantity of smoking cessation compound absorbed by limiting the amount of time spent chewing the mass of chewing gum. In some of these embodiments the period of time is between 2 minutes and 15 minutes.

In various preferred embodiments the mass of chewing gum can be reattached to the distal end of the shaft after it has been removed from the distal end of the shaft and chewed by the smoker. And in some preferred embodiments further include portion indicators that facilitate separate removal from the distal end of the shaft of indicated portions of the mass of chewing gum.

In preferred embodiments the mass of chewing gum further includes a flavoring ingredient.

In some preferred embodiments the device further includes a removable and reusable cover that is able to protect the mass of chewing gum from contamination when the chewing gum is attached to the distal end of the shaft.

Another general aspect of the invention is a device for helping a tobacco user, herein referred to as a “smoker,” to quit the use of tobacco, herein referred to as “smoking.” The device includes a shaft having a length and shape that allow a smoker to conveniently hold a proximal end of the shaft in one hand of the smoker while placing a distal end of the shaft in the smokers' mouth, the shaft including a hollow interior that is open at the distal end, and a mass of chewing gum contained within the hollow interior of the shaft. The mass of chewing gum is extendable beyond the distal end of the shaft for removal and chewing by the smoker, and contains a quantity of a smoking cessation compound in a form suitable for absorption into the smoker's bloodstream through tissues within the mouth of the smoker.

In preferred embodiments, the smoking cessation compound is one of nicotine and a nicotine substitute. Some preferred embodiments include portion indicators that facilitate separate removal from the distal end of the shaft of indicated portions of the mass of chewing gum. And In certain preferred embodiments the mass of chewing gum further includes a flavoring ingredient.

In other preferred embodiments the device further includes a removable and reusable cover that is able to protect the mass of chewing gum from contamination. In some of these embodiments the shaft and the removable and reusable cover include a cover attachment mechanism that is able to fix the removable and reusable cover to the distal end of the shaft so as to protect the mass of chewing gum from contamination.

In various embodiments the device further includes a chewing gum delivery mechanism that is able to move the mass of chewing gum toward the distal end of the shaft, thereby causing a portion of the mass of chewing gum to extend beyond the distal end of the shaft. In some of these embodiments the chewing gum delivery mechanism includes a protrusion extending through a slot in a wall of the shaft, the protrusion being cooperative with the mass of chewing gum and movable along a length of the slot so as to cause the mass of chewing gum to move toward the distal end of the shaft. In other of these embodiments the chewing gum delivery mechanism includes an outer cylinder surrounding a portion of the length of the shaft, the outer cylinder being cooperative with the mass of chewing gum so as to cause the mass of chewing gum to move toward the distal end of the shaft when the outer cylinder is rotated relative to the shaft.

Yet another general aspect of the present invention is a kit for helping a tobacco user, herein referred to as a “smoker,” to quit the use of tobacco, herein referred to as “smoking.” The kit includes a plurality of nicotine chewing gum delivery devices. Each of the nicotine chewing gum delivery devices includes a shaft having a length and shape that allow a smoker to conveniently hold a proximal end of the shaft in one hand while placing a distal end of the shaft in the smoker's mouth, and a mass of chewing gum cooperative with the distal end of the shaft. The mass of chewing gum is removable from the distal end of the shaft, and contains a quantity of a smoking cessation compound in a form suitable for absorption into the smoker's bloodstream through tissues within the mouth of the smoker.

Each of the nicotine chewing gum delivery devices has a unique combination of a chewing gum flavor, an amount of chewing gum included in the mass of chewing gum, an amount of smoking cessation compound, and a rate of absorption of the smoking cessation compound into tissues of the mouth of a smoker when the smoker chews the mass of chewing gum.

### BRIEF DESCRIPTION OF THE DRAWINGS

The invention will be more fully understood by reference to the detailed description, in conjunction with the following figures, wherein:

FIG. 1A is a perspective view of a preferred embodiment that includes a spherical mass of nicotine chewing gum attached to the end of a stick;

FIG. 1B is a perspective view of the preferred embodiment of FIG. 1A that includes a paper wrapper covering the chewing gum;

FIG. 1C is a perspective view of the embodiment of FIG. 1A, showing the device in use;

FIG. 1D is a perspective view of a preferred embodiment that includes a cylindrical mass of nicotine chewing gum divided into segments and attached to a stick;

FIG. 2A is a perspective view of a preferred embodiment of the invention that includes a hollow shaft filled with nicotine chewing gum and a cover attached to the shaft so as to cover the chewing gum;

FIG. 2B is an perspective view of the embodiment of FIG. 2A shown with the cap removed;

FIG. 2C is a perspective view of the embodiment of FIG. 2A, showing the device in use;

FIG. 3A is a perspective view of an embodiment similar to FIG. 2A but including a clip for securing the cover to a shaft;

FIG. 3B is a partially cut-away view of the embodiment of FIG. 3A, showing the nicotine chewing gum beneath the cover;

FIG. 3C is a perspective view of the embodiment of FIG. 3A, showing the cover removed from the end of the shaft from which the gum extends and installed for temporary storage on the other end of the shaft;

FIG. 4 is a perspective view of an embodiment similar to FIG. 2A and FIG. 3A but including a shirt pocket clip on the cover, a sliding mechanism for advancing the chewing gum out from the interior of the shaft, and a connecting chain that secures the cover to the shaft; and

FIG. 5 is an exploded view of a preferred embodiment that includes a hollow shaft containing nicotine chewing gum and rotatable mechanism that advances the chewing gum out of the hollow shaft with a twisting movement similar to a typical lipstick.

### DETAILED DESCRIPTION OF PREFERRED EMBODIMENTS

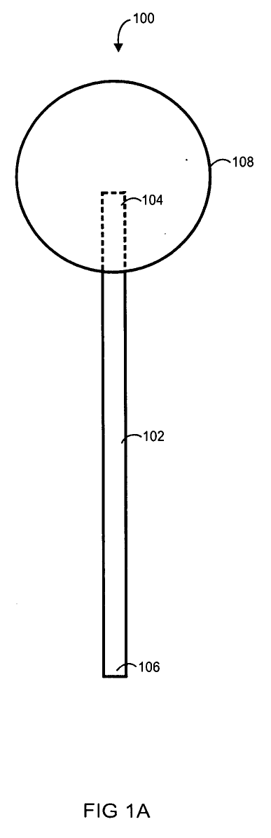

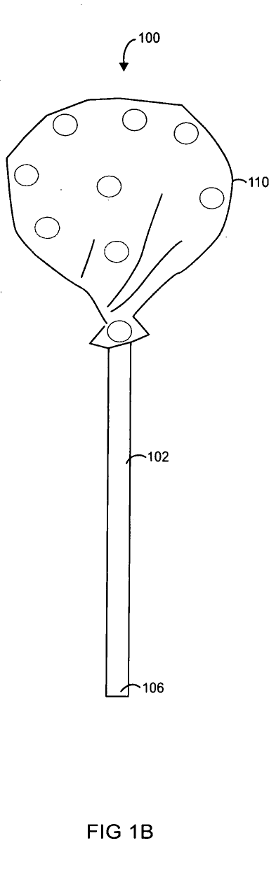

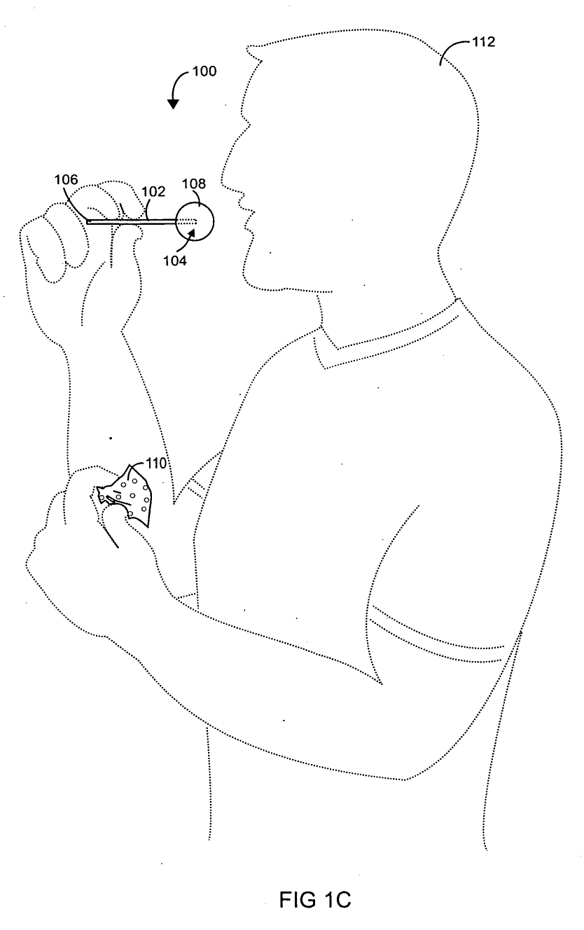

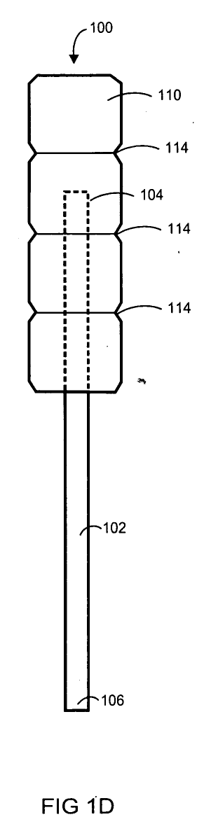

FIG. 1A illustrates a preferred embodiment of the present invention. The device 100 includes a stick or a shaft 102 having a distal end 104 and a proximal end 106, with a spherical mass of nicotine chewing gum 108 attached to the distal end 104 of the shaft. FIG. 1B illustrates the embodiment of FIG. 1A, in which a paper cover 110 is included and is illustrated as covering the mass of chewing gum so as to protect it from contamination when the chewing gum not in use. FIG. 1C illustrates the embodiment of FIG. 1A and FIG. 1B being used by a a tobacco user 112, herein referred to as a “smoker,” 112. The cover 110, protecting the mass of chewing gum 108 from contamination, has been removed and is reusable.

The shaft 102 is sized so as to allow the smoker 112 to conveniently hold the proximal end 106 of the shaft while the distal end 104 is in the mouth of the smoker 112. In this embodiment, when the smoker 112 is finished chewing the mass of chewing gum 108, the mass of chewing gum 108 can be re-attached to the distal end 104 of the shaft 102, thereby allowing the smoker 112 to control the timing and the dosage of nicotine or nicotine substitute delivered.

The mass of chewing gum 108 contains a smoking cessation compound, such as nicotine or a nicotine substitute, in a form suitable for absorption into the bloodstream of the smoker 112 through tissues within the mouth of the smoker 112. By limiting the amount of time spent in chewing the mass of chewing gum 108, the smoker 112 is able to limit the quantity of smoking cessation compound absorbed. In preferred embodiments that use nicotine as the smoking cessation compound, the amount of nicotine contained in the mass of chewing gum 108 is preferably between 1 and 3 micrograms. The rate of absorption of the smoking cessation compound also varies between different embodiments, and preferably allows for full absorption of the compound during a chewing time of between 2 and 15 minutes. The mass of chewing gum 108 may further include a flavoring ingredient, such as a mint flavor for example.

FIG. 1D shows a perspective view of an embodiment similar to the embodiment of FIG. 1A, except that the mass of chewing gum 108 is cylindrical and is divided into segments by indentations 114 in the chewing gum 108. The indentations 114 allow a smoker 112 to more easily bite off a desired quantity of the chewing gum 108, and to keep track of the quantity of chewing gum that has already been chewed.

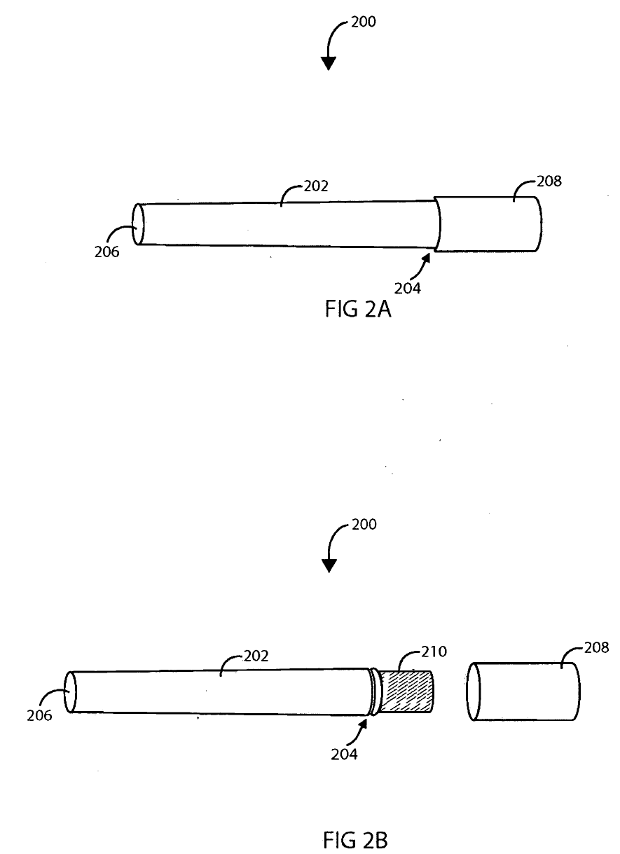

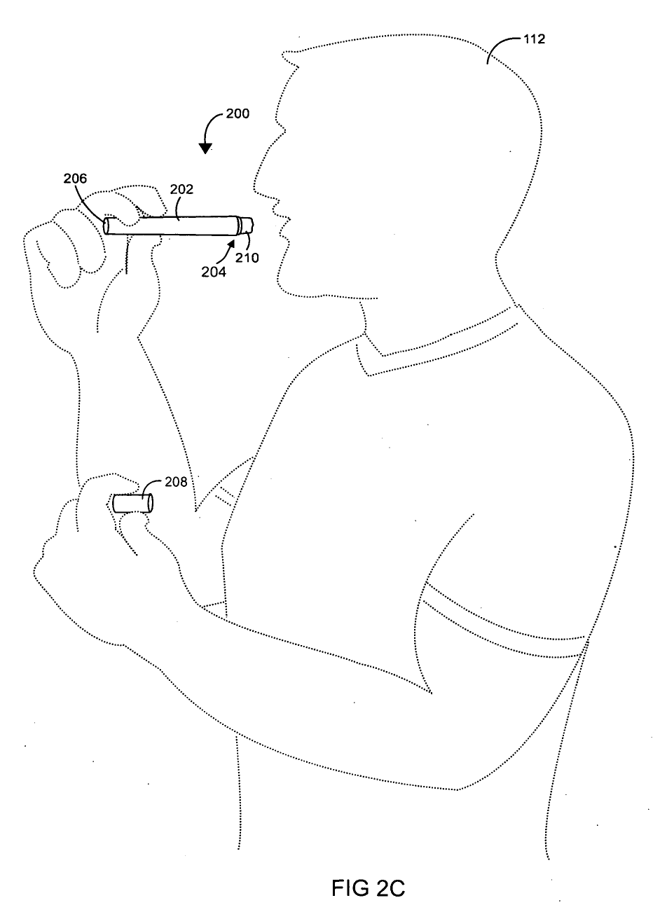

FIG. 2A is a perspective view of a preferred embodiment 200 that includes a shaft 202 that has a distal end 204, and a proximal end 206, with a cover 208 attachable to the distal end 204 of the shaft 202. FIG. 2B illustrates the embodiment of FIG. 2A with the cover removed, and FIG. 2C illustrates the embodiment 200 of FIG. 2A in use. When using this embodiment, a smoker 112 places the distal end 204 of the shaft 202 inside his or her mouth and bites off a desired quantity of the nicotine chewing gum 210. In the embodiment of FIG. 2A, the shaft 202 is hollow, and is able to contain a quantity of the chewing gum 210. The inner lining of the shaft 202 is made from a material that does not adhere to a used mass of chewing gum 210, thereby allowing chewing gum to be chewed and then conveniently replaced onto the end of the shaft for later re-use. Materials used in preferred embodiments include, but not limited to, Teflon, specially treated metals, wood, glass, injection molded polymers, and the like.

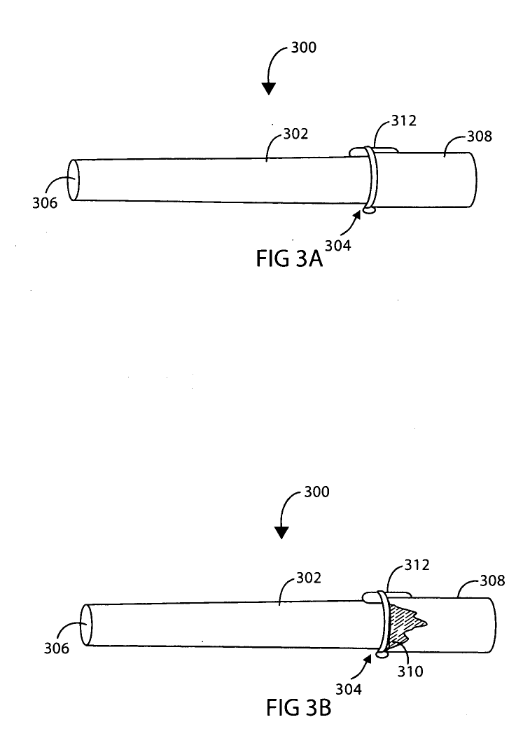

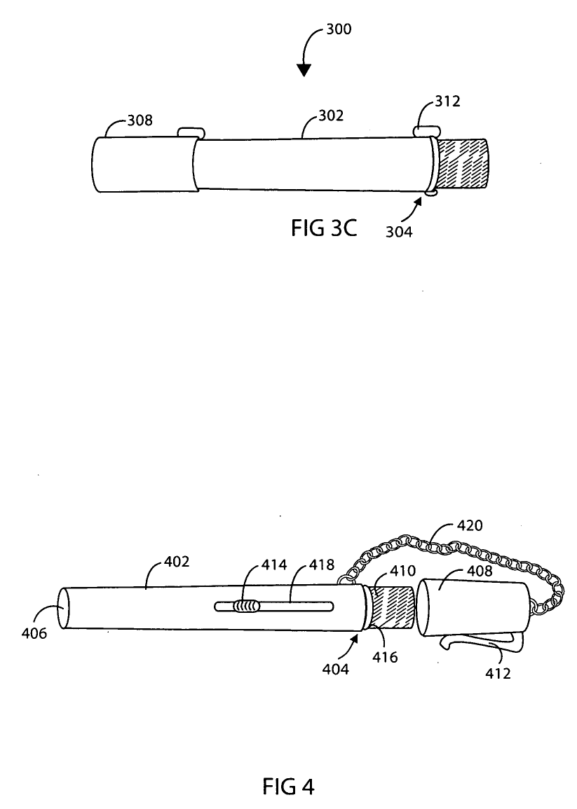

FIG. 3A is a perspective view of another embodiment 300 of the present invention that is similar to the embodiment of FIG. 2A but includes a clip 312 that holds the cover 308 firmly secured to a shaft 302. FIG. 3B is a partially cut-away view of the embodiment of FIG. 3A showing the location of nicotine chewing gum 310 beneath the cover 308. FIG. 3C is a perspective view of the embodiment 300 showing the cover 308 attached for temporary storage to the proximal end 306 of the shaft 302.

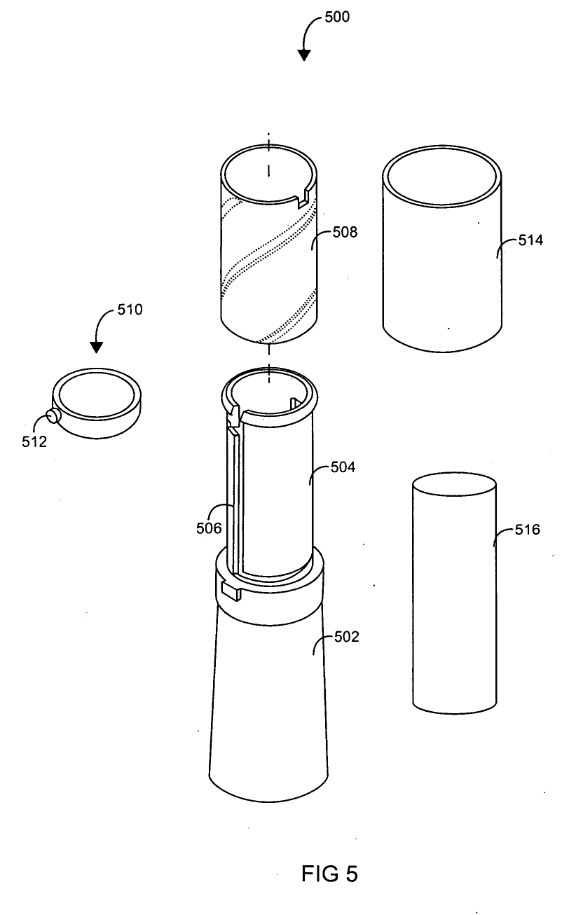

FIG. 4 is a perspective view of a still another embodiment 400 of the present invention that is similar to the embodiments of FIG. 2A and FIG. 3A, but includes a shirt pocket clip 412 on the cover 408 and a sliding mechanism for advancing nicotine gum 410 out of the shaft. The sliding mechanism includes a protrusion 414 that slides along a slot 418 so as to push the nicotine chewing gum 410 beyond the distal end 404 of the shaft 402.

In the embodiment 400 of FIG. 4 the cover 408 is secured to the shaft 402 by a connecting member 420 to prevent loss of the cover 408. In FIG. 4 the connecting member is illustrated as a chain, while in similar embodiments the connecting member 420 can be made from string, rope, wire, or any other, suitable material.

FIG. 5 is an exploded view of a preferred embodiment that includes a mechanism that extends nicotine chewing gum 516 from the hollow center of the shaft 504 in a manner similar to a typical lipstick. The device 500 includes a base 512 and an upright tube 504 with an upright slot 506. The shaft 500 includes a rotary tube 508 that is rotatable about the upright tube 504 and a chewing gum mount 510 movably disposed in the upright tube 504. The chewing gum mount 510 is provided with a projection 512 which is received in the upright slot 506 of the upright tube 504. The shaft 500 further includes an outer tube 514 fitted over the upright tube 504, and a cover 516 fitted over the outer tube 514 and covering the shaft 500.

In some preferred embodiments, a kit is provided for helping a smoker 112 to quit the use of tobacco products. The kit includes a plurality of smoking cessation devices according to embodiments of the present invention such as the embodiments described above 100, 200. The devices in the kit vary in at least one aspect, such as the flavor of the chewing gum, the concentration of smoking cessation compound contained in the chewing gum, the quantity of chewing gum attached to the shaft, and/or the rate of absorption of the smoking cessation compound as controlled by its formulation. By making a selection from among the devices in the kit, a smoker 112 is thereby enabled to select a preferred flavor and/or a preferred quantity and/or rate of dosage of the smoking cessation compound according to the smoker's needs and preferences at the time of usage.

Other modifications and implementations will occur to those skilled in the art without departing from the spirit and the scope of the invention as claimed. Accordingly, the above description is not intended to limit the invention, except as indicated in the following claims.

What is claimed is:

1. A device for helping a tobacco user, herein referred to as a “smoker,” to quit the use of tobacco, herein referred to as “smoking,” the device comprising:
a shaft having a length and shape that allow a smoker to conveniently hold a proximal end of the shaft in one hand while placing a distal end of the shaft in the smoker's mouth; and
a mass of chewing gum cooperative with the distal end of the shaft, the mass of chewing gum being removable from the distal end of the shaft, the mass of chewing gum containing a quantity of a smoking cessation compound in a form suitable for absorption into the smoker's bloodstream through tissues within the mouth of the smoker.

2. The device of claim 1, wherein the smoking cessation compound is one of nicotine and a nicotine substitute.

3. The device of claim 1, wherein the quantity of smoking cessation compound is at most sufficient to alleviate nicotine withdrawal symptoms to a degree comparable to a dosage of nicotine received by smoking a cigarette.

4. The device of claim 1, wherein the smoking cessation compound is formulated and configured such that it will be absorbed over a period of time into the bloodstream of the smoker as the smoker chews the mass of chewing gum, so as to enable the smoker to limit the quantity of smoking cessation compound absorbed by limiting the amount of time spent chewing the mass of chewing gum.

5. The device of claim 4, wherein the period of time is between 2 minutes and 15 minutes.

6. The device of claim 1, wherein the mass of chewing gum can be reattached to the distal end of the shaft after it has been removed from the distal end of the shaft and chewed by the smoker.

7. The device of claim 1, further including portion indicators that facilitate separate removal from the distal end of the shaft of indicated portions of the mass of chewing gum.

8. The device of claim 1, further including a flavoring ingredient.

9. The device of claim 1, further comprising a removable and reusable cover that is able to protect the mass of chewing gum from contamination when the chewing gum is attached to the distal end of the shaft.

10. A device for helping a tobacco user, herein referred to as a “smoker,” to quit the use of tobacco, herein referred to as “smoking,” the device comprising:
a shaft having a length and shape that allow a smoker to conveniently hold a proximal end of the shaft in one hand of the smoker while placing a distal end of the shaft in the smokers' mouth, the shaft including a hollow interior that is open at the distal end; and
a mass of chewing gum contained within the hollow interior of the shaft and extendable beyond the distal end of the shaft for removal and chewing by the smoker, the mass of chewing gum containing a quantity of a smoking cessation compound in a form suitable for absorption into the smoker's bloodstream through tissues within the mouth of the smoker.

11. The device of claim 10, wherein the smoking cessation compound is one of nicotine and a nicotine substitute.

12. The device of claim 10, further including portion indicators that facilitate separate removal from the distal end of the shaft of indicated portions of the mass of chewing gum.

13. The device of claim 12, further including a flavoring ingredient.

14. The device of claim 10, further comprising a removable and reusable cover that is able to protect the mass of chewing gum from contamination.

15. The device of claim 14, wherein the shaft and the removable and reusable cover include a cover attachment mechanism that is able to fix the removable and reusable cover to the distal end of the shaft so as to protect the mass of chewing gum from contamination.

16. The device of claim 10, further comprising a chewing gum delivery mechanism that is able to move the mass of chewing gum toward the distal end of the shaft, thereby causing a portion of the mass of chewing gum to extend beyond the distal end of the shaft.

17. The device of claim 16, wherein the chewing gum delivery mechanism includes a protrusion extending through a slot in a wall of the shaft, the protrusion being cooperative with the mass of chewing gum and movable along a length of the slot so as to cause the mass of chewing gum to move toward the distal end of the shaft.

18. The device of claim 16, wherein the chewing gum delivery mechanism includes an outer cylinder surrounding a portion of the length of the shaft, the outer cylinder being cooperative with the mass of chewing gum so as to cause the mass of chewing gum to move toward the distal end of the shaft when the outer cylinder is rotated relative to the shaft.

19. A kit for helping a tobacco user, herein referred to as a “smoker,” to quit the use of tobacco, herein referred to as “smoking,” the kit comprising a plurality of nicotine chewing gum delivery devices, each nicotine chewing gum delivery device including:
a shaft having a length and shape that allow a smoker to conveniently hold a proximal end of the shaft in one hand while placing a distal end of the shaft in the smoker's mouth; and
a mass of chewing gum cooperative with the distal end of the shaft, the mass of chewing gum being removable from the distal end of the shaft, the mass of chewing gum containing a quantity of a smoking cessation compound in a form suitable for absorption into the smoker's bloodstream through tissues within the mouth of the smoker;
each of the nicotine chewing gum delivery devices having a unique combination of:
a chewing gum flavor;
an amount of smoking cessation compound
an amount of chewing gum included in the mass of chewing gum; and
a rate of absorption of the smoking cessation compound into tissues of the mouth of a smoker when the smoker chews the mass of chewing gum.
